(19) United States
(12) Patent Application Publication
(10) Pub. No.: US 2023/0346566 A1
(43) Pub. Date: Nov. 2, 2023

(54) Prosthesis, and Associated Methods of Implanting A Joint Replacement and Implanting A Prosthetic Bone Joint Replacement as For Ankle Replacement With Press-Fit Tibia Component, Spherical Articulation and Method of Implantation

(71) Applicant: Kamran Syed Hamid, Chicago, IL (US)

(72) Inventor: Kamran Syed Hamid, Chicago, IL (US)

(21) Appl. No.: 18/307,538

(22) Filed: Apr. 26, 2023

Related U.S. Application Data
(60) Provisional application No. 63/335,303, filed on Apr. 27, 2022.

(51) Int. Cl. A61F 2/42

(52) U.S. Cl. CPC A61F 2/4202; A61F 2310/00011; A61F 2002/4205; A61F 2002/4207

(57) ABSTRACT

Prosthetic ankle replacements, prostheses, and associated methods of implanting prosthetic ankle and other joint replacements. In one form, a tibia stem has a geometry to facilitate implant installation while providing means to transfer load over a larger surface area through a “press-fit,” “scratch-fit” or “cortical fit”, thus lowering the contact stresses at the tibia-metal implant interface. The design and its implantation procedure also address minimization of misalignment. A talus element presents a partial-hemispherical surface; a tibial element which is to be affixed within the tibia, having an elongated stem part which extends into and within a canal of the tibial inner cortex, an intermediate body part which smoothly widens in radius toward an end which is a base to the tibial element, the base having a concavity formed therein shaped to match the partial-hemispherical surface of the talus element with a plastic cap between the two.

### CROSS-REFERENCE TO RELATED APPLICATIONS

This application claims the benefit of U.S. Provisional Application No. 63/335,303, filed Apr. 27, 2022, the contents of which are incorporated herein by reference.

### BACKGROUND OF THE DISCLOSURE

The invention generally relates to prostheses and associated surgical procedures, including prosthetic replacements, prostheses, and associated methods of implanting a prosthetic replacement and implanting a prosthetic bone joint replacement. The invention relates in particular to ankle replacement prostheses, systems and associated surgical procedures/methodology. Aspects of the invention may have applications beyond the ankle.

A healthy ankle joint is composed of three bones (the tibia, talus and fibula) which are shown in their standard spatial arrangement in FIGS. 1A and 1B. Soft tissue components of the ankle such as ligaments, cartilage, muscles, tendons, and synovial fluid, are not shown in this illustration.

The ankle joint is between the foot and the lower leg. The ankle joint is highly complex, as it carries nearly all of an individual's body weight, while allowing the foot to move upwardly (dorsiflexion), downwardly (plantarflexion), side to side (inversion and eversion) as well as twisting movement. The lower leg connection to the ankle is made up of the tibia 10 and fibula 12, which engage with the talus 14. The bottom of the talus 14 sits on the calcaneus, which is the heel bone.

FIGS. 1A and 1B show a typical healthy ankle joint with its bones in their standard healthy spatial arrangement. Ligaments hold the ankle joint together. The talus 14 may be considered to essentially nest within the ankle (FIG. 1A) socket with the two leg bones, the tibia 10 and fibula 12. In addition to the ankle, there are two critical joints presented which assist in motion of the foot with relationship to the leg. There is a subtalar joint which allows “inversion” and “eversion” of the foot. Finally, there is the talonavicular joint, which provides shock absorption and assists in dorsiflexion, plantarflexion, inversion, eversion and twisting.

In ideal circumstances, the natural ankle joint can last a lifetime without significant pain or dysfunction. The lifespan of a functional ankle can be reduced, however, by age-related degeneration, trauma, instability, misalignment and/or inflammatory disease, to name but a few potential ankle issues. These conditions damage the cartilage layers that protect the ends of articulating bones, very often resulting in bone-on-bone engagement—a condition known as “arthritis.” Arthritis is a painful condition which typically results in limiting function of the ankle.

Bones in their normal state have an envelope of hard cortical bone 16 surrounding the softer inner trabecular bone 18, as illustrated in FIG. 2. The mechanical strength of long bones like the tibia varies over its cross-section, with the outer cortical part having higher strength than the interior trabecular part. There is a gradual transition from the middle shaft portion of the tibia to the distal (lower) portion at the level of the ankle so when the leg carries a mechanical load (weight of the person), the bones shrink or compress in the axial direction and expand or bulge a smaller amount in the radial direction. As a result, the effective inner volume will decrease with a resultant increase in pressure inside the cortical envelope. Thus, the axial load-carrying-capacity of a bone is inherently reduced, as when it is cut or resected. For instance, and as related to the instant disclosure regarding total ankle replacement (TAR), when the surgery is performed, the load capacity of the effected bones is reduced, but expectedly only in a transient situation, after the TAR is initially installed. If the tibia has appropriate healing and ingrowth to the metallic implant components, the enclosed cortical outer shell state can be effectively reestablished.

Ankle fusion is another approach for treating ankle degeneration, but it has serious consequences in loss of ankle mobility. It involves either an external or internal fixation of the ankle after removal of cartilage. In external fixation, surgical pins are fixed inside the leg and ankle bones to keep the bones in place until they fuse. In an internal fixation approach, the ankle and leg bones are compressed with internal plates and screws so that the bones fuse.

Cortical and trabecular bones undergo stress and strain property changes as part of the aging process. This behavior is different from inorganic engineering materials where reversible mechanical properties may change acutely in response to temperature and pressure (or stress) but not significantly due to time. Mismatched intrinsic properties between bone and metal can result in problems at their interface, but also provide opportunities for improved prosthesis design.

TAR is used to reduce pain and improve function in patients with ankle arthritis. There are currently about twelve TARs reported to be approved for use in the United States at the time of this disclosure:
Scandinavian Total Ankle Replacement (STAR™) (WO).
Hintermann Series H3™ Total Ankle Replacement System (DT MedTech).
Inbone II™ Total Ankle System (Wright Medical)
Infinity™ Total Ankle System (Wright Medical).
Invision™ Total Ankle Revision System (Wright Medical).
Cadence® Total Ankle System (Integra LifeSciences).
Salto Talaris® Ankle (Integra LifeSciences).
Trabecular Metal™ Ankle Replacement (Zimmer Biomet).
Vantage® Total Ankle System (Exactech),
Apex 3D™ Total Ankle Replacement System (Paragon28).
Quantum® Total Ankle System (IN2BONES).
Kinos Axiom™ Total Ankle System (Kinos Medical/Restor3d).

The Hintermann Series H3 and Scandinavian Total Ankle Replacement (STAR) are two “mobile-bearing,” “3-piece,” or “unconstrained” TAR systems. They have a metal tibia component, metal talus component and an intermediary plastic component that is curved on the talus side and flat on the tibia side. The plastic component is unconstrained, and not connected to either of the metallic parts; thus, there are three distinct pieces that can “move” with respect to each other. Other TARs have similar design except they are “constrained,” “two-piece” or “fixed” since the plastic component is attached to the tibia component. Regardless of fixed or unconstrained, all modern TARs have basically similar designs as demonstrated in FIGS. 3A and 3B.

FIGS. 3A and 3B depict a typical standard conventional TAR design. The conventional TAR design is composed of a metal component 20 attached to the talus (a “talus element”), a metal component 24 attached to the tibia end (a “tibial element”), and a hard plastic component 22 that interfaces between the metal talus element 20 and the metal tibial component 24. This serves to illustrate the limited contact surface area provided compared to the natural bone engagements.

There are minor differences in how the metal components attach to bone. However, there are notable differences in two systems as depicted in FIGS. 4A and 4B. FIG. 4A shows the INBONE II system, which has a similar arrangement of talus metal component 26, plastic intermediate element 28, and metal tibial component 30. Here, the metal tibial component 30 also includes a stem element 32. The metal tibial component 30 is attached to the tibia. The metal talus element 26 is attached to the talus. The plastic component is disposed between the tibia component 30 and the talus element 26. The stem 32 is not press-fit into the tibial cortex.

FIG. 4B illustrates the Trabecular Metal TAR system, which is similarly composed of a metal talus element 34, an intermediate plastic component 36, and a metal tibial element 38. The metal tibial element 38 is a curved metal component attached to the tibia. The metal talus element 34 is attached to the talus. The intermediate plastic component 36 is a curved plastic component between the tibia element 38 and the talus element 34. Noteworthy is the cylindrically curved nature of all contacting surfaces of the three components 34, 36, and 38. During installation, standard conventional TAR approaches have the talus and tibia cut to make provisions to install the TAR components.

FIG. 5A shows the kind of box cuts C that are used to emplace the tibial component of a common TAR. The high stress riser corners of the box cut C present interior to the cut out. FIG. 5B is a schematic cross section showing how the box cut C of the tibia results in edge loading of the tibia at E. Regardless of their differences, all existing TARs have relatively similar functions and limitations.

Typically, the TAR implant (also called a prosthesis, prosthetic, or similar terms) itself is made of standard biocompatible metal, hard plastic (e.g., UHMWPE), and/or ceramic engineering materials. Standard TAR approaches have the talus and tibia cut to make provisions to install the TAR components. Of course, the engineering materials do not possess the characteristics of natural living bones and cartilage, although many try to approximate the same properties, but do not provide all the functionality that is required to meet the joint performance that nature has provided. Also, TAR selection and installation are dependent on the experience level and technical ability of the surgeon. The TAR system, while it is reasonably successful in minimizing pain, generally has a relatively low operational or full functional duration life (often less than one to up to about ten years), and can require multiple revision procedures. The early failures (i.e., less than one year) are generally due to problems in the installation or post-surgery treatment process, which can require re-installation surgeries. The high end of the TAR life (e.g., 10 years) is still not considered sufficient in high demand (active) patients and those with significant life remaining. As noted, the alternative to ankle replacement is an ankle fusion surgery. This has numerous downsides that make it unpalatable for many patients. As patients desiring ankle replacements skew younger and have higher physical demands, the standard current TARs are insufficient in matching these needs.

There are various limitations associated with existing total ankle replacements, some of which are summarized below.

Limited contact area at bone-metal interface and plastic-metal interface leads to high stresses: The contact area of TAR (FIGS. 5A and 5B) is far less than that of the native ankle (FIGS. 1A and 1B), resulting in high stresses and accelerated wear that potentially requires revision surgery. Wear particles released in use can also result in damage to the ankle in some cases.

Stress risers: Bone machining (cutting) leaves sharp corners in the load-bearing structures of the tibia (FIGS. 5A and 5B). These sharp corners result in high localized stresses and can lead to material failure. Cracks may form in the cortical bone due to its higher brittleness. These cracks can cause leakage of fluid from the joint resulting in bone cysts, fracture propagation or implant failure.

Alignment: The natural ankle joint is inherently tolerant to misalignment of bones, but the replaced ankle joint is not as forgiving. This is due to the replacement of the natural pseudo-spherical joint with a cylindrical joint limiting the degrees of freedom for relative motion of the bones. There is little to no means provided in the standard existing designs to prevent/minimize the effects of misalignment. Its life under the conditions of boundary lubrication is relatively short (around 5-10 years as observed in the industry.) Small amounts of misalignment result in line contact with edge-loading, and subsequent accelerated wear.

Plastic deformation: In existing designs, modifications of the tibia and talus are made to interface bone with metallic and hard plastic components of a TAR. These changes result in reduced capacity of the tibia to carry both static and dynamic weights of the patient. FIG. 5A shows a cut section of the tibia in preparation for a TAR. Parts of the cortical bone along with some parts of the trabecular bone have been cut. Since a significant part of the cortical bone has been removed, the mechanical compressive stresses due to the patient's weight may increase beyond the compressive strength of the bone. As a result, these high stress areas will undergo plastic deformation which will loosen the joint resulting in premature failures (10-30%) of TARs. This scenario may change when the bones are completely healed with the formation of cortical bones in areas enclosing the trabecular parts fully. The stress level would vary depending upon the body mass index (BMI) of the patient and contact area of the implant with bone. For patients with normal BMI, the stress levels may be close to the yield point and hence it will subside at a slow rate resulting in the anticipated approximately ten years of TAR life. Given the present lifespan in the US is around 84 years, and the prostheses are generally installed between ages 40-70 years, ideally a desired TAR life needs to be much higher than ten years.

Limited ability to tolerate high forces: The material properties and design of the TAR components play a significant role in the performance of the TAR. The failure mechanisms observed in the industry indicate that the TAR components appear to survive well with low impact activity, however, subsidence of the bones appears to be the primary mode of failure as discussed above. Additionally, patients are limited in activity. High impact activity may result in accelerated failure of the TAR.

The mechanical force transferred from the upper part of the tibia to the lower part of the tibia is done predominantly through the outer cortical bone. Since parts of the original cortical bone are removed, stress levels may increase beyond the yield point, resulting in plastic deformation. This effect will also occur in the talus where the top surface is cut-off to make room for the metallic piece thereby exposing the softer trabecular structure. Like the tibia, subsidence of the talus surface can be a slow process, taking 5-10 years, though early subsidence can occur in patients with poor talus bone quality or misalignment.

Limited inversion and eversion: Existing TARs allow for an “up and down” movement of the ankle called dorsiflexion and plantarflexion. There is limited “side to side” movement called inversion and eversion due to the cylindrical joint restricting the degree of motion discussed earlier. Inversion and eversion motion comes from the subtalar joint underneath the ankle and the ankle itself. In cases of ankle arthritis, there is often concomitant subtalar arthritis as well. Thus, most patients with ankle arthritis have limited inversion and eversion. Replacing the ankle joint with an existing TAR does not aid in inversion or eversion.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

Design and methodology rationale: The proposed design and related implementation method seeks to overcome existing deficiencies in the replacement by creating robust contact between, in a presently preferred application, a tibia stem and the inside surface of the tibia cortical bone. This allows improved axial load transfer with lower stress levels imposed on the tibia and talus. FIGS. 6A and 6B schematically illustrate this aspect. (Note, the features needed to attach the plastic component to the tibia component are not shown for simplicity.)

The inventive design, in one form, has a tibia stem geometry that has been optimized to facilitate implant installation while providing improved means to transfer load over a larger surface area (“press-fit,” “scratch-fit” or “cortical fit”), thus lowering the contact stresses at the tibia-metal implant interface. The design and its implantation procedure also address minimization of misalignment. Assembly tools have been contemplated and/or adapted and developed to result in proper alignment (see various of FIGS. 7A through 7Q).

The present invention includes, in one non-limiting form, a first member anchored to the tibia and a second member anchored to the talus, and operably associated with the first member (see FIGS. 6A and 6B). An intermediate member, such as a hard plastic component, may preferably be included to act as a sliding interface between the first and second members.

The shape of the implant stem, or tibial component, in one form has been optimized for increased contact area based on the inner geometry of the tibia's cortical bone. A long stout stem provides radial stiffness and prevents high frequency cyclic loads from reaching the sliding joint and thus preventing micromotion. The tibia stem may be cylindrical or contoured to match the inner cortical bone. Contouring may be by custom-design, as by an Additive Manufacturing process for one example.

The new design also eliminates the sharp corners in the design to prevent stress magnification at corners. Corners have been preferably rounded to minimize stress concentration. In a preferred form, the implant stem is monolithic (one piece), without need of any mechanical fixation within a channel in the tibia end other than a press fit. Additional fixation as by use of cement may nonetheless be considered.

In an embodiment, the present TAR stem is placed by making a large resection oblique bone cut to give enough space to place the implant. There is precedence for this in the hip joint where a large resection oblique cut is used to help place a press-fit stem. The hip joint has excellent large contact areas for force transfer and is the most resilient and successful joint replacement in the body. However, there have been no studies that the present Inventor is aware of in the ankle demonstrating bone preservation as advantageous for TAR life as compared to a larger resection. Oftentimes, the quality of distal tibia bone is poor due to the juxtaposed arthritis. Making such a translation from hip joint to ankle joint is thus not precedented.

Another advantageous feature in one form of the new design is conversion of the cylindrical joint (of the current design) to the spherical (ball and socket) type joint. The ball and socket arrangement will inherently disperse load over a wide area, and hence reduce the stress level which should result in longer bearing life similar to those of hip and knee joint replacements.

In a preferred form, the implant and its methodology have also been made to maximize the contact area of the joint by partially cutting into the ends of tibia and fibula (again, see FIGS. 6A and 6B) without compromising the ligaments. This further reduces the stress levels experienced in tibia and talus, reducing the propensity to subside while improving the bearing life. The syndesmosis articulation (between tibia and fibula) is essentially non-functional in TAR for two reasons. First, the syndesmosis is generally arthritic in conjunction with the arthritic ankle joint. Second, after placement of a TAR the lateral gutter is typically cleared out to prevent impingement of the talus with the fibula. Thus, there is minimal to no anterior to posterior force on the fibula, and it will not move. The decision was made herein to span the tibia-fibula articulation with the implant and utilize this non-functional space.

Improved assembly: Assembly tools and procedures have been introduced to improve alignment and preload accuracy on the stem to distribute the load over a large area depicted variously in FIGS. 7A-7Q. The technique of osteotomy, canal entry and sequential broaching mimics the standard technique for hip replacement femoral stem placement, but it is considered to be new and unconventional as provided to ankle replacement. In order to place the tibia component, a cavity is gradually created through controlled broaching to accept the top surface of the stem. Broaching and tibia device placement may be done manually or using a computer-controlled system with load sensors for another technique.

Long life: It is expected that keeping stresses below the endurance limit will result in enhanced life. Improved alignment also helps with longevity. Nanomaterials may be utilized in the parts design to improve boundary lubrication. Microscopic passages may be provided through the upper stem and the top side to allow fluid migration to lubricate the sliding surfaces (see FIG. 9).

In an embodiment, the design process for a tibial stem involves obtaining the geometry of the tibia. From the geometry of the tibia bone's external surface an estimate of the internal cortical part of the bone could be obtained. See, e.g., Lauren E. Jansen, Nathan P. Birch, Jessica D. Schiffman, Alfred J. Crosby, and Shelly R. Peyton, “Mechanics of Intact Bone Marrow”, J Mech Behav Biomed Mater, 842 (2015).

From the inside geometry of the tibia, a tentative shape of the stem may be generated. This shape can be analyzed for its loadbearing capacity. If found to be sufficiently strong, a graphical means (or computerized equivalent means) could be used to determine the ability to assemble the stem into the tibia. During assembly, known processes could be incorporated to measure the applied axial force to calculate the measured radial interference. From the bone properties, the adequacy of the interference may be evaluated.

Manufacturing of TAR components: While not exclusive, it is considered most preferred that all metallic components of the inventive TAR (i.e., stem and talus connector) are made by a sintering method wherein powdered material (of graded particle size distribution) is used to make a part in ‘green state’ by compressing the powder in a mold. The green part is then heated in a furnace to the sintering temperature and held for a required length of time. The sintered metal and plastic materials have lower mechanical strength but are stronger than the bones. They also have a lower Young's modulus (compared to the parent material) which tends to make their compressive behavior similar to that of bones for a given size. This helps distribute load more uniformly over the contact area between the tibia and talus. Manufacturing may also be done by 3D printing (Additive Manufacturing) to achieve similar characteristics. Standard coating and porous surfaces may also be created to allow for bone ingrowth or cement fixation.

The spherical joint between the disclosure's hard plastic component and talus metallic component may be made with projections at the periphery of the plastic component to slow escape of synovial fluid during axial shock loading. Based on the amount of fluid between the components, these projections may or may not be necessary.

There are numerous advantages in the presented disclosure of implant new design and methodology, some or all of which may be incorporated in embodiments:

Large articulation contact area: TAR life will be increased due to larger contact area for load transfer from tibia to talus through the spherical joint. The fluid pressure (or equivalently the mechanical stress) in the bearing area is reduced due to the larger contact area (see FIGS. 6A and 6B). The lower stress level should increase the boundary layer thickness which should reduce the wear rate in the sliding parts.

Decreased mechanical subsidence of the tibia metal component: Subsidence is reduced due to the larger contact area from the press-fit tibia stem component as compared to the traditional end-loading tibia components (FIGS. 3A, 3B, 5B).

Improved Performance: The new TAR should allow a patient to perform higher impact activities as compared to traditional TARs.

Temperature Reduction: The reduced friction and increased mass in the TAR joint will lead to lower transient temperatures (in the sliding joint) keeping it below 42° C., and thus reducing the degradation of synovial fluid and preventing solids precipitation from the synovial fluid at high transient temperatures (e.g., T>42° C.) in the sliding joint.

Ball-Socket Joint: Design of the new TAR joint is based on a spherical (ball and socket) joint allowing angular displacements of the required magnitudes about two orthogonal axes (see FIGS. 6A and 6B). This makes the design more tolerant to angular misalignment of tibia to talus, preventing wear due to edge loading. It also provides protection against shock loads. Additionally, the spherical design allows for inversion and eversion movement to further offload adjacent joints.

Press-fit Stem: The length and diameter of the stem will be designed to result in a press-fit which will ensure required load transfer capability through the cortical section of healthy proximal tibia. The tibia component will not rely on arthritic distal tibial bone for support.

Cushioning effect: Articulation will be designed to maintain a sufficient boundary layer for cushioning between the plastic and metal talus components during axial shock loading. A plastic component may advantageously include peripheral projections to retain sufficient synovial fluid between components.

In one preferred form, a prosthetic ankle replacement of the disclosure has a talus element which is to be affixed to the surface of the talus, the talus element presenting a partial-hemispherical surface. A tibial element is to be affixed within the tibia, the tibial element having an elongated stem part which extends into and within a canal of the tibial inner cortex, an intermediate body part which widens in radius toward an end which is a base to the tibial element, the base having a concavity formed therein shaped to match the partial-hemispherical surface of the talus element.

The tibial element is adapted to conform to a shape formed within the tibia presenting tibial cortex canal sidewalls so as to be press fit into the tibial cortex canal, and most preferably affixed in place without further means of fixation. The prosthesis may further include a hard plastic cap component which provides an interface between a talus element partial-hemispherical surface and a tibial base concavity.

In an aspect, the disclosure contemplates a prosthetic for implantation in a joint of a human or animal, the joint including a bone having a cortical canal. This prosthesis has a stem part which has a long axis and a radial aspect, the stem part having a distal end which extends into and within the canal of the canal in use, and an intermediate body part which widens in radius toward a proximal end which is a base to the stem part.

The stem part is adapted to present a stem exterior that conforms to a shape formed in the canal yielding an open canal interior sidewall which is matched to the radius of the stem. The stem part is press fit into the canal and held in place through tight contact between the canal interior sidewall and the stem exterior. Most preferably, the stem part is affixed without any use of cement or other mechanical fixation.

The foregoing prosthetic may further include a first element which is to be affixed to the surface of a first end of a joint, the first element presenting a shape upon which the stem part will articulate, such as a partial-hemispherical surface.

The intermediate body part which widens in radius toward an end which is the base that will articulate on the first element. For one example, the base has a concavity formed therein shaped to match the partial-hemispherical surface of the first element.

One method according to the disclosure is a method for implantation of a prosthetic joint replacement. A prosthesis is provided of a first element which is to be affixed to the surface of an end of a first bone of the joint, the first element in one form presenting a partial-hemispherical surface. A second element is to be affixed within a cortical canal of a second bone of the joint, the second element having an elongated stem part which extends into and within a canal of the cortical canal, an intermediate body part which widens in radius toward an end which is a base to the second element, and in one form, the base having a concavity formed therein shaped to match the partial-hemispherical surface of the first element.

The method includes cutting an end of the second bone to expose the inner canal, and successively widening the canal through removal of intramedullary bone therein so as to match the external shape of the second element.

Then affixing the first element to the surface of the first bone so as to present the partial-hemispherical surface toward the concavity of the second element. The second element is press fit into the canal in tight engagement with a sidewall forming the interior of the canal. Then the first element is seated within the concavity of the second element. The foregoing method is most preferably wherein the second element is affixed without any use of cement or other mechanical fixation.

The method may further include providing a hard plastic cap component which provides an interface between the first element partial-hemispherical surface and the base concavity.

In a preferred application, the first element is adapted for a talus element, and the stem part is adapted for a tibial element which is received within a canal of a tibial inner cortex.

A preferred form of the invention has the stem part formed in a single piece, as by an Additive Manufacturing process, which can then be further customized for the patient.

According to yet another nonlimiting aspect of the invention, a prosthesis for implantation in a joint of a human or animal includes a first element configured to be affixed to a surface of a first end of the joint with the first element presenting an articulation surface, and a second element configured to be affixed within a second end of the joint opposed to the first end. The second element includes an elongated stem part configured to extend into and within a canal of the second end of the joint, an intermediate body part which widens in radius toward an end which is a base to the second element. The base has a shape formed therein shaped to match the articulation surface of the first element. The second element is adapted to conform to a shape formed in the canal so as to be press fit into the canal.

According to still another nonlimiting aspect of the invention, a prosthesis for implantation in a joint of a human or animal includes a stem part which has a long axis and a radial aspect. The stem part has a distal end which extends into and within the cortical canal in use, and an intermediate body part which widens in radius toward a proximal end which is a base to the stem part. The stem part is adapted to present a stem exterior that conforms to a shape formed in the cortical canal yielding an open canal interior sidewall that is matched to the radius of the stem, so as to be press fit into the cortical canal and held in place through contact between the canal interior sidewall and an exterior of the stem.

According to a still further nonlimiting aspect of the invention, a method for implantation of a prosthetic bone joint replacement is provided. The method includes providing a prosthesis that includes a first element which is to be affixed to the surface of an end of a first bone of the joint, the first element presenting a support surface, and a second element which is to be affixed within a cortical canal of a second bone of the joint, the second element having an elongated stem part which extends into and within the cortical canal, an intermediate body part which widens in radius toward an end which forms a base to the second element, the base having a shape formed therein shaped to match the support surface of the first element. The method also includes cutting an end of the second bone to expose the cortical canal, successively widening the cortical canal through removal of intramedullary bone therein so as to match an external shape of the second element, press fitting the second element into the cortical canal into tight engagement with a sidewall forming an interior of the cortical canal, affixing the first element to the surface of the first bone so as to present the support surface toward the second element, and seating the first and second element.

In some arrangements, relative to conventional total join replacements, and in particular conventional total ankle replacement prostheses, the apparatus and methods of the present may provide for larger articulation contact area, decreased mechanical subsidence of the tibia metal component, improved performance, temperature reduction, ball-socket joint functionality, press-fit stem coupling, and/or a cushioning effect.

These and other aspects, arrangements, features, and/or technical effects and advantages and aspects will be further understood upon consideration of the following description of certain embodiments, taken in conjunction with the drawings.

### BRIEF DESCRIPTION OF THE DRAWINGS

FIGS. 1A and 1B are an anterior and lateral views, respectively, of the bones making up a human ankle. FIG. 1A shows normal ankle joint bones, viewing the right ankle from the front (anterior view). FIG. 1B shows the normal ankle joint bones, viewing the right ankle from the side (lateral view).

FIG. 2 is a schematic axial cross-sectional view through the tibia.

FIGS. 3A and 3B are anterior and lateral schematic views of a typical standard prior art TAR design.

FIG. 4A is a lateral schematic view of a prior art INBONE II TAR system.

FIG. 4B is a lateral schematic view of a prior art Trabecular Metal TAR design.

FIG. 5A is a schematic anterior view of cuts made to the tibial end to receive a standard conventional TAR tibial implant component.

FIG. 5B is a cross sectional schematic view illustrating a distal tibia cut of a standard conventional TAR.

FIG. 6A is a schematic anterior view of an embodiment of a total replacement ankle according to nonlimiting aspects of the present invention.

FIG. 6B is a lateral view of the TAR shown in FIG. 6A.

FIG. 6C is an enlarged cross-sectional view of the TAR of FIG. 6A demonstrating articulation between components of the TAR at the metal talus and plastic dome interface.

FIGS. 7A-7Q illustrate various steps in a procedure for implanting the prostheses of FIG. 6A in a human ankle joint to form a total ankle replacement.

FIG. 7A is an anterior view of an ankle with the tibial inner cortex and tibial outer cortex shown prior to being altered for implantation of a TAR.

FIG. 7B is an anterior view of the ankle that illustrates large drill holes used to create an anterior cortex window.

FIG. 7C is a lateral view of the ankle that illustrates the drill paths from the lateral view.

FIG. 7D is a lateral view of the ankle that illustrates a curved awl used to initially penetrate the tibial canal.

FIG. 7E is a lateral view of the ankle that shows the curved awl as used to create a path for broaches in the tibial canal.

FIG. 7F is a lateral view of the ankle that illustrates how a small broach is placed in the tibial canal as part of a sequentially larger broaching process.

FIG. 7G is a lateral view of the ankle that shows a larger broach placed in the tibial canal with press-fit.

FIG. 7H is a lateral view of the ankle that schematically shows a jig is attached to the final broach to allow for burr or saw resection of the distal tibia medially and the distal fibula laterally.

FIG. 7I is an anterior view of the ankle that illustrates the way a cutting tool would be proposed to be used to create distal tibia medial resection and distal fibula resection.

FIG. 7J is an anterior view of the ankle that illustrates the tibia after resection of bone.

FIG. 7K is a schematic lateral view of the tibia and the talus after resection of bone from both.

FIG. 7L is a schematic lateral view of the tibia and the talus that illustrates plantarflexion of the talus to provide space for the tibial component.

FIG. 7M is a schematic lateral view of the tibia and the talus that illustrates an initial insertion of a tibial component in the tibia according to a nonlimiting example of the present invention.

FIG. 7N is a schematic lateral view of the tibia and the talus that illustrates deeper placement of the tibial component of FIG. 7M into the tibia.

FIG. 7O is a schematic lateral view of the tibia and the talus that shows the tibial component as press-fit in place in the tibia.

FIG. 7P is a schematic lateral view of the tibia and the talus that shows the completed TAR after implantation and insertion of the plastic interface component and metal talus element.

FIG. 7Q is an anterior view of the ankle with the TAR in FIG. 7P with the dotted line representing the anterior cortex window.

FIGS. 8A and 8B show another form of a talus component which has one or more radial extensions which reduce possible excessive inversion or eversion.

### DETAILED DESCRIPTION OF EMBODIMENTS

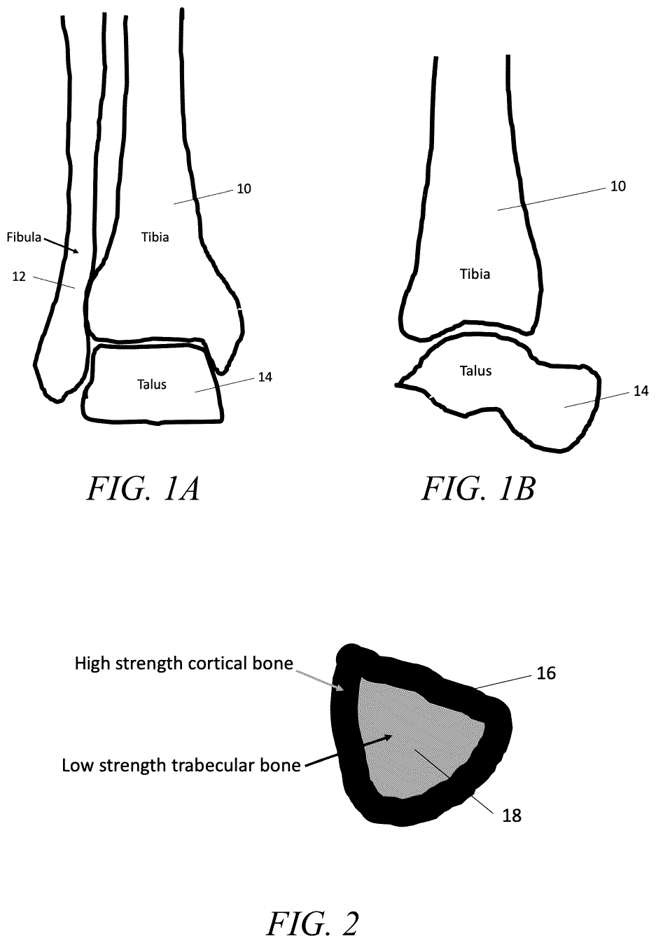

Turning now to the drawings, FIGS. 1A and 1B illustrate bones commonly associated with the ankle: tibia 10, fibula 12 and talus 14. In FIG. 2, a schematic cross section of the tibia 10 illustrates the cortical bone 16 surrounding the lower strength interior trabecular bone 18.

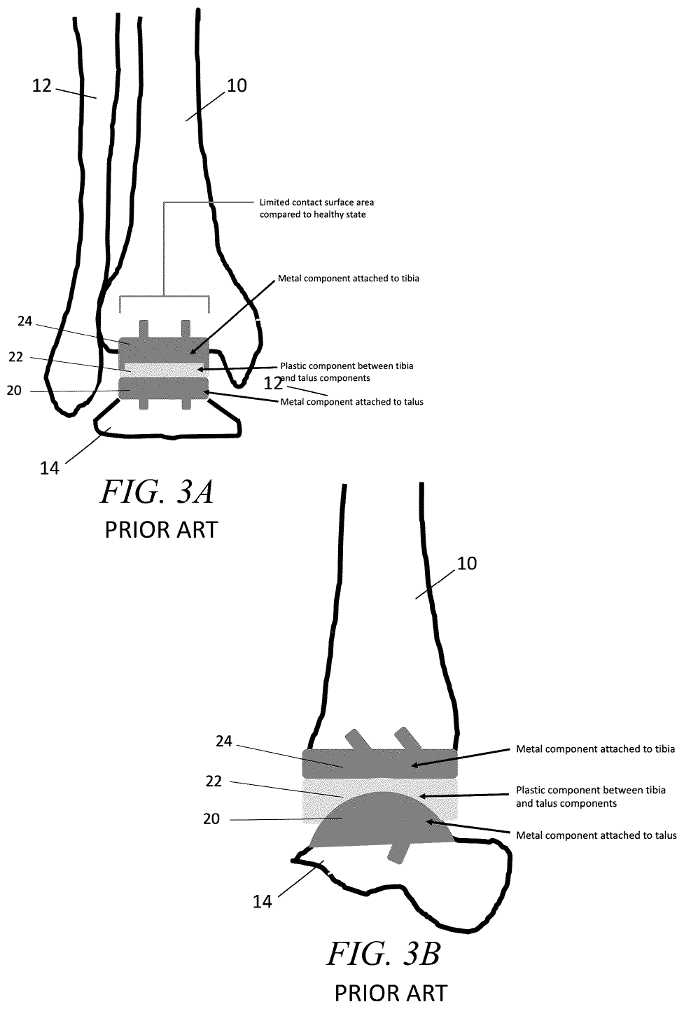

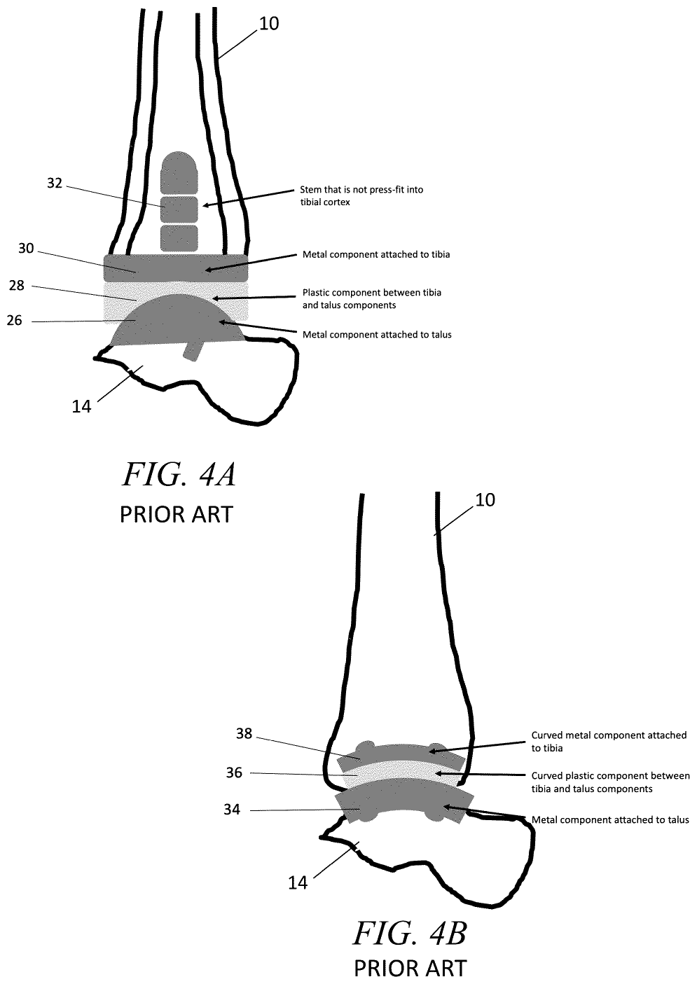

Some prior art TAR system components are illustrated in FIGS. 3A-3B and FIGS. 4A and 4B. The FIGS. 3A and 3B drawings are intended to depict a “standard” TAR design. It is composed of a metal component 20 attached to the talus, a hard plastic component 22 that interfaces between the talus metal component 20 and a metal component 24 attached to the tibia end. This serves to illustrate the limited contact surface area provided compared to the natural bone engagements.

In the so-called INBONE II system, shown in FIG. 4A, there is a similar arrangement of talus metal component 26, plastic intermediate element 28 and metal tibial component 30. Here, the latter also includes a stem element 32.

The so-called Trabecular Metal TAR system is illustrated in FIG. 4B. Here again, it is similarly composed of a metal talus element 34, an intermediate plastic component 36 and a tibial metal component 38. Noteworthy is the cylindrically curved nature of all contacting surfaces.

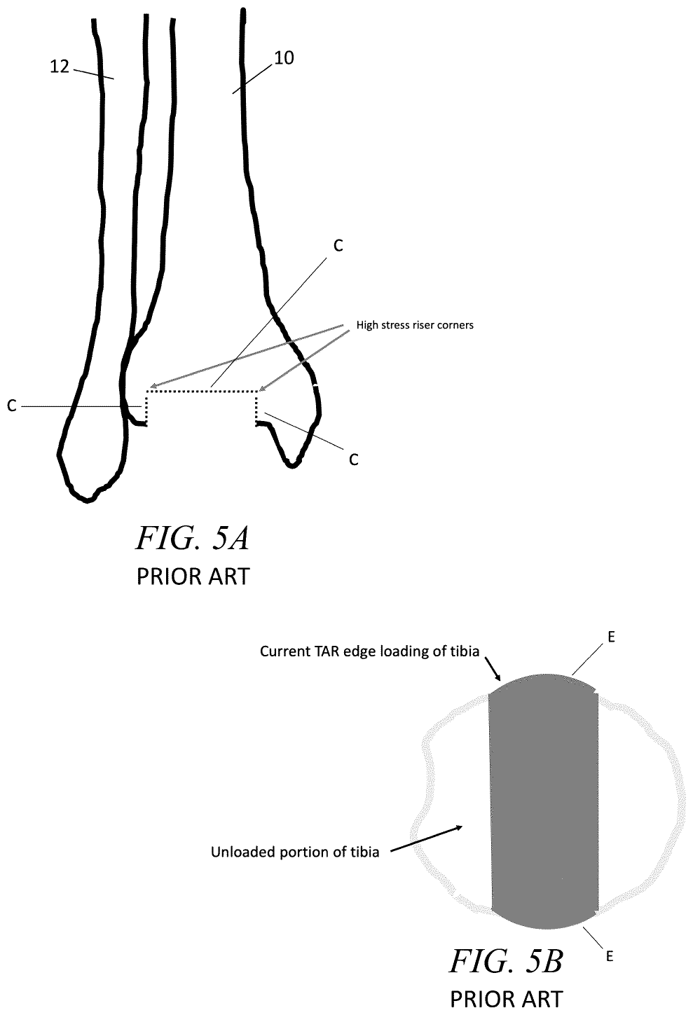

FIG. 5A shows the kind of box cuts C that are used to emplace the tibial component of a common TAR. Note the high stress riser corners present interior to the cut out. FIG. 5B is a schematic cross section showing how such a cut of the tibia results in edge loading of the tibia at E.

The intended purpose of the following detailed description of the invention and the phraseology and terminology employed therein is to describe what is shown in the drawings, which include the depiction of and/or relate to one or more nonlimiting embodiments of the invention, and to describe certain but not all aspects of the embodiment(s) to which the drawings relate. The following detailed description also identifies certain but not all alternatives of the embodiment(s). As nonlimiting examples, the invention encompasses additional or alternative embodiments in which one or more features or aspects shown and/or described as part of a particular embodiment could be eliminated, and also encompasses additional or alternative embodiments that combine two or more features or aspects shown and/or described as part of different embodiments. Therefore, the appended Claims, and not the detailed description, are intended to particularly point out subject matter regarded to be aspects of the invention, including certain but not necessarily all of the aspects and alternatives described in the detailed description.

Although the invention will be described hereinafter in reference to the ankle and total ankle replacement shown in the drawings, it will be appreciated that the teachings of the invention are more generally applicable to a variety of types of bone joints in the body.

To facilitate the description provided below of the embodiment(s) represented in the drawings, relative terms, including but not limited to, “proximal,” “distal,” “anterior,” “posterior,” “vertical,” “horizontal,” “lateral,” “front,” “rear,” “side,” “forward,” “rearward,” “top,” “bottom,” “upper,” “lower,” “above,” “below,” “right,” “left,” etc., may be used in reference to the orientation of the TAR during its use and/or as represented in the drawings. All such relative terms are useful to describe the illustrated embodiment(s) but should not be otherwise interpreted as limiting the scope of the invention.

According to certain nonlimiting aspects of the invention, a TAR has a tibia stem geometry that has been optimized to facilitate implant installation while providing improved means to transfer load over a larger surface area (“press-fit,” “scratch-fit” or “cortical fit”), thus lowering the contact stresses at the tibia-metal implant interface. The design of the TAR components and the implantation procedure for the TAR also address minimization of misalignment. Assembly tools have been contemplated and/or adapted and developed to result in proper alignment.

According to other certain nonlimiting aspects of the invention, a prosthetic ankle replacement includes a member anchored to the tibia (also called a “stem,” “tibial implant” or “tibial element”) and a member anchored to the talus (also called a “talus implant” or “talus element”). The members are operably associated. An intermediate member, such as a hard plastic component, may preferably be included to act as a sliding interface between the tibial and talus elements.

In one nonlimiting example, the shape of the stem of the tibial component is optimized for increased contact area based on the inner geometry of the tibia's cortical bone. A long stout stem provides radial stiffness and prevents high frequency cyclic loads from reaching the sliding joint and thus preventing micromotion. The stem of the tibial implant may be cylindrical or contoured to match the inner cortical bone. Contouring may be by custom-design, as by an Additive Manufacturing process for one example.

According to other certain nonlimiting aspects of the invention, the TAR eliminates the sharp corners in the design to prevent stress magnification at corners. Corners have been preferably rounded to minimize stress concentration. In a preferred form, the implant stem (tibial element) is monolithic (one piece), without need of any mechanical fixation within a channel in the tibia end other than a press fit. However, additional fixation, such as by use of cement, may nonetheless be considered.

According to other certain nonlimiting aspects of the invention, the TAR stem (tibial element) is implanted in the tibia by making a large resection oblique bone cut to give enough space to place the tibial implant. There is precedence for this in the hip joint where a large resection oblique cut is used to help place a press-fit stem. However, the inventor is not aware of any studies in the ankle demonstrating bone preservation as advantageous for TAR life as compared to a larger resection. Oftentimes, the quality of distal tibia bone is poor due to the juxtaposed arthritis. Making such a translation from hip joint to ankle joint is thus not precedented.

According to other certain nonlimiting aspects of the invention, an advantageous feature in one form of the present TAR design is conversion of the cylindrical joint (of the conventional design) to a spherical (ball and socket) type joint in the present TAR. The ball and socket arrangement will inherently disperse load over a wide area, and hence reduce the stress level which should result in longer bearing life similar to those of hip and knee joint replacements.

According to yet other certain nonlimiting aspects of the invention, the implant and its methodology preferably are configured to maximize the contact area of the joint by partially cutting into the ends of tibia and fibula without compromising the ligaments. This further reduces the stress levels experienced in tibia and talus, reducing the propensity to subside while improving the bearing life. The syndesmosis articulation (between tibia and fibula) is essentially non-functional in TAR for two reasons. First, the syndesmosis is generally arthritic in conjunction with the arthritic ankle joint. Second, after placement of a TAR the lateral gutter is typically cleared out to prevent impingement of the talus with the fibula. Thus, there is minimal to no anterior to posterior force on the fibula, and it will not move. The present TAR spans the tibia-fibula articulation with the implant and utilizes this non-functional space.

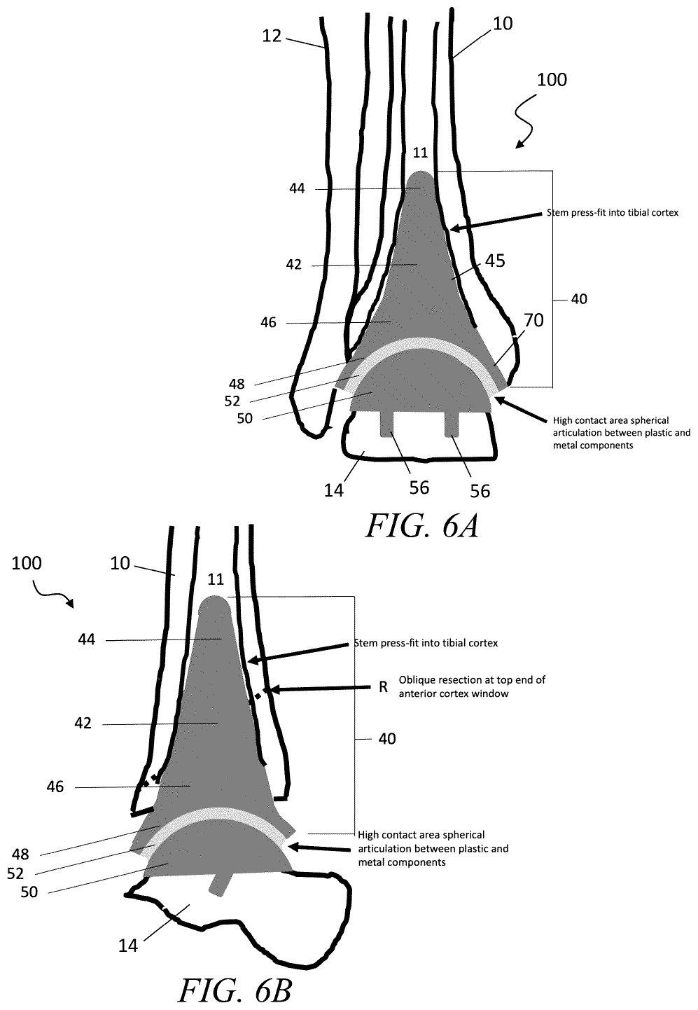

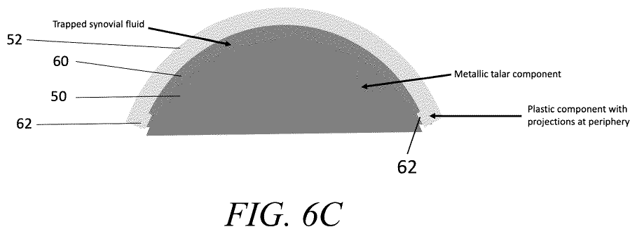

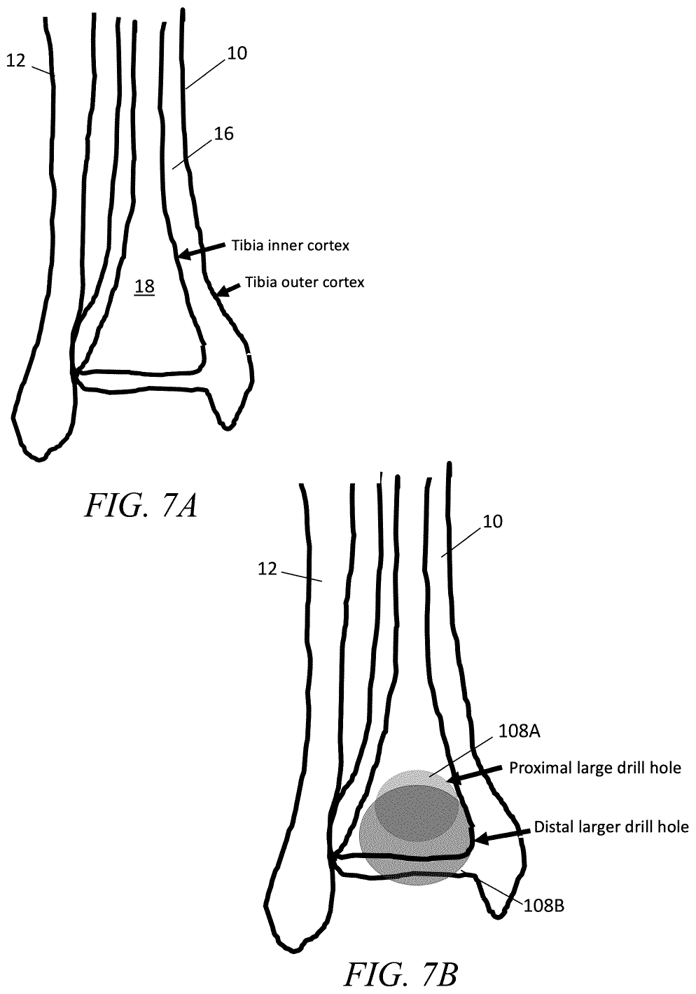

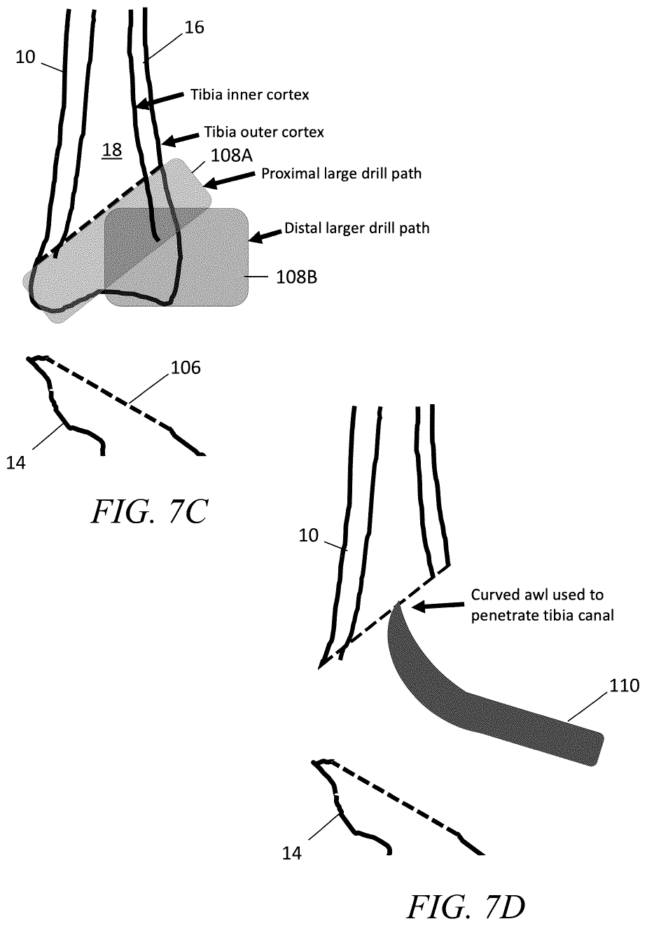

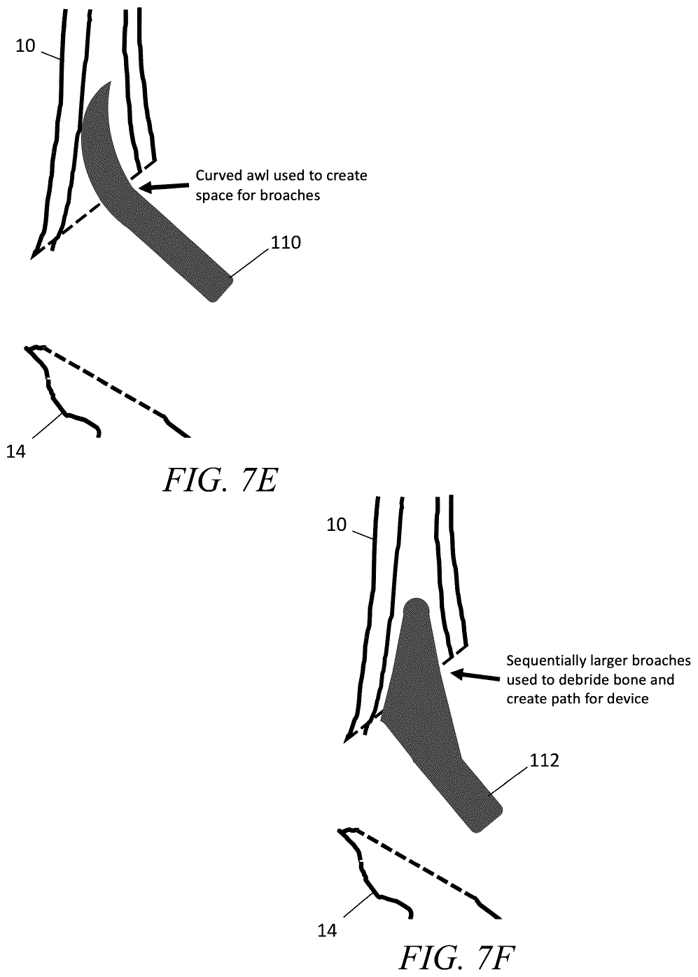

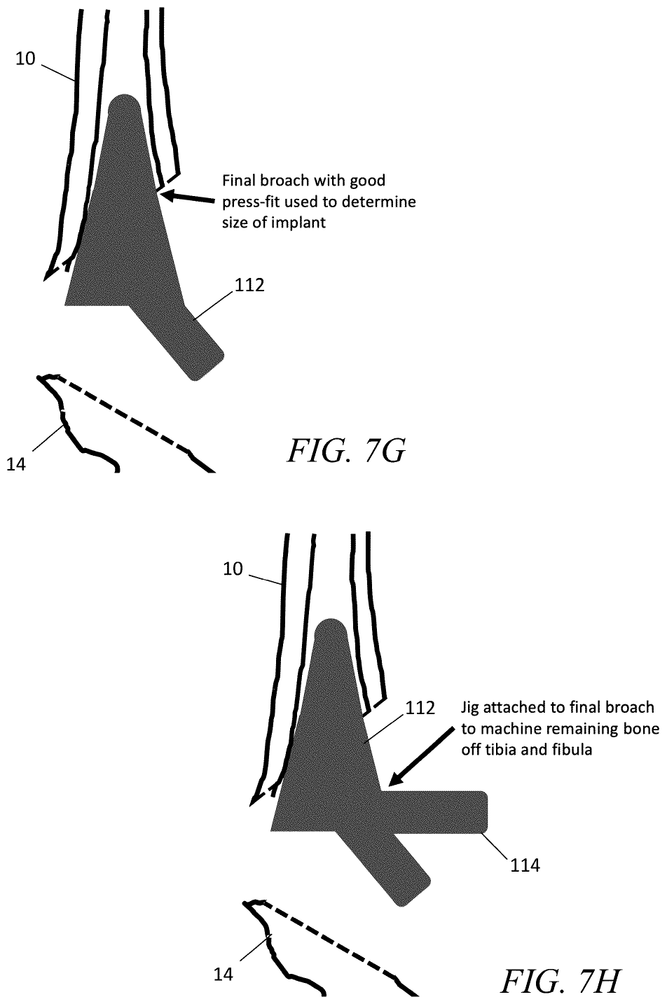

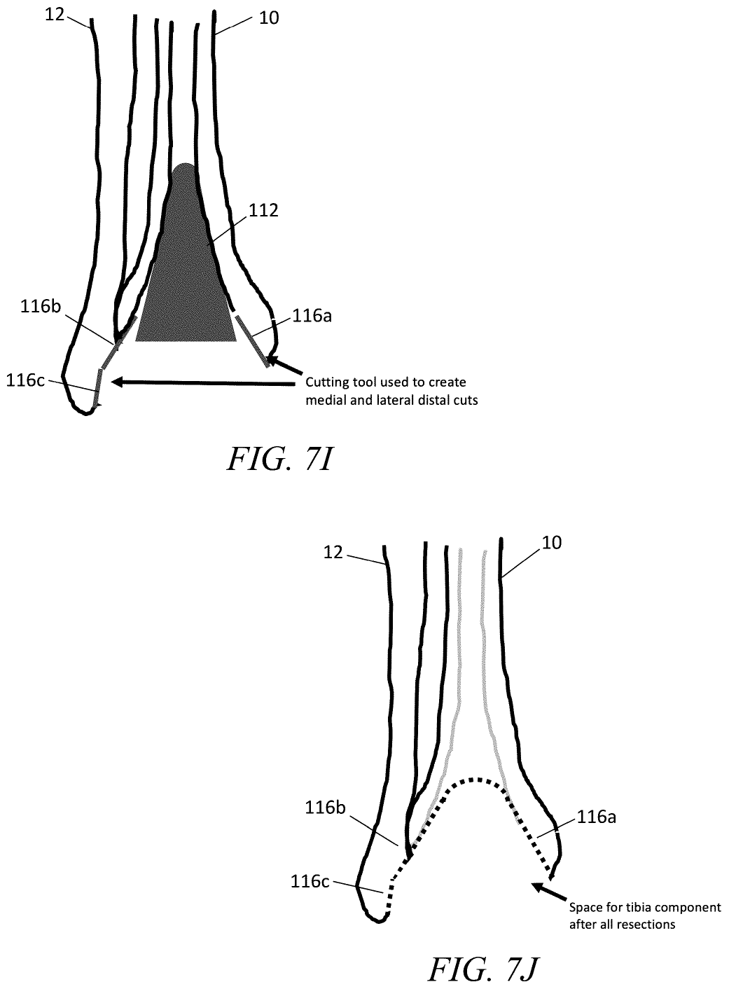

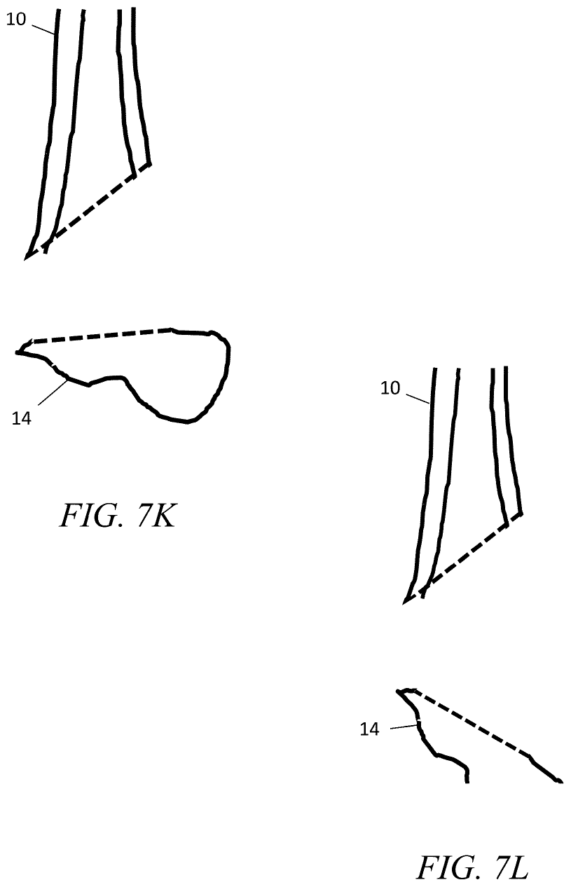

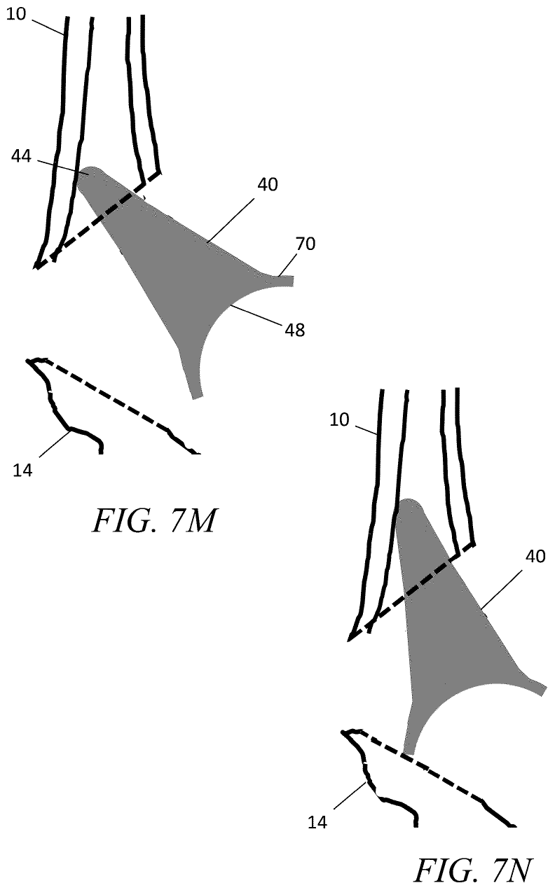

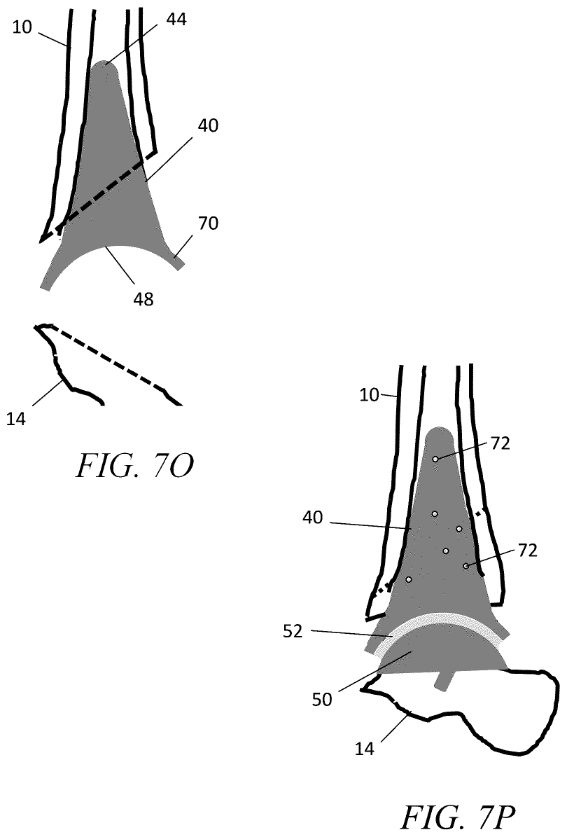

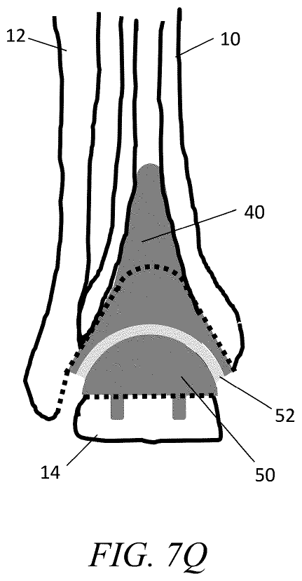

Turning now to the nonlimiting example in the drawings, FIGS. 6A-7Q depict certain examples and aspects of a TAR and associated prosthetic components and methods of installing the TAR according to various principles of the invention. However, the invention is not necessarily limited to TARs and/or use only with human ankle joints, but rather may also be applied to other bone joints in humans and/or other animals.

FIGS. 6A and 6B show an embodiment of a TAR 100 (also called a prosthetic ankle replacement, a prosthesis, a prosthetic bone joint replacement, and similar terms throughout). The TAR 100 includes an upper prosthetic component (referred to herein as a tibial element or stem part) 40 that attaches to the distal end of the tibia 10, a lower prosthetic component (referred to herein as a talus element or talus part 50) that attaches to an upper surface of the talus 14, and a plastic interface component (liner) 52 that is disposed between and engages against the stem part 40 and the talus part 50. Preferably, each of the stem part 40 and the talus part 50 is made of a suitable hard bio-compatible metal (e.g., stainless steel, titanium, alloys, etc.) such that the liner 52 forms a soft(er) seating surface between the opposing joint surfaces of stem part 40 and the talus part 50.

As shown in FIGS. 6A and 6B, in one embodiment, the upper prosthetic component or stem part 40 comprises an elongated tibial stem part 42 which extends from one end 44 that will be located within the tibial intramedullary canal 11, and widens in radius to an opposite end 46 forming a base to the stem part 40. There is a spherical concavity 48 formed in the base, which as will be seen, engages with a complimentary spherical convexity of the liner 52 and metal talus components. The talus components comprise a solid talus hemispherical shaped element 50 and inset within the concavity 48 is in the liner 52, which is preferably a hard plastic. These provide a high contact area spherical articulation between the plastic and metal components. While set forth in this embodiment as being metal, the tibial stem part 40 may be made of any material or materials commonly used in the prosthetic arts, including, but not limited to, metals, ceramics, or any other total joint replacement metal and/or ceramic, bony in-growth surface, including such things as artificial bone, uncemented metal or ceramic surface, or a combination thereof. The tibial stem may further be covered with one or more coatings such as antimicrobial, antithrombotic, and osteoinductive agents, or a combination thereof.

The tibial stem part 40 may be variable lengths, and variable widths. In the preferred embodiment, the tibial stem has a flared shape extending from a nose inserted up into the tibial intramedullary canal along a smooth widening radius to its interface with the talus part 50 and plastic liner componentry. It should be understood that the disclosed tibial stem could be of virtually any length, depending upon the size of the patient, his or her bone dimensions, and the anticipated future mobility of the patient. A custom-made tibial component is most preferably considered. The tibial stem is inserted into the reamed intramedullary passage superiorly through the tibia.

For example, as depicted in FIGS. 7D, 7E, 7F and 7G a passage using an awl and then successive reaming devices superiorly through the tibia, to form a space matching the shape of the intended tibial element, such that the latter can be press fit into place. The stem may further be fixed in the lower tibia as with polymethylmethacrylate bone cement, hydroxyapatite, a ground bone composition, or any other fixation materials common to one of skill in the art of prosthetic surgery.

Note that the base of the stem has a concavity formed therein, which is matched to the talus componentry which has a convexity such that the stem component and concentric plastic liner will smoothly fit on the talus element.

As shown in FIGS. 6A, 6B, 7P and 7Q, the talus portion of an embodiment of the present invention has a curved or dome shape presenting toward the tibia. This talus part would be preferably formed as a single piece, including pegs 56 that affix in holes drilled into the talus in known fashion. The pegs can be placed in different positions on the talus component than depicted, and alternatively, the talus component could have slots to accept screws, staples, pins, or any other fixation devices. The pegs or slots can be part of the hemispherical talus component or part of a projection extending off the component. Thus, the talus part 50 is fixed to the top of the talus. It may be made of various materials commonly used in the prosthetic arts including, but not limited to metals, alloys, ceramic, or any other joint replacement metal and/or ceramic, including most preferably surface bone engaging the talus component.

In this embodiment, the liner (interface component) 52 is provided between the talus part and the tibial stem and forms a part of the talus componentry. This interface is preferably ultra-high molecular weight polyethylene (UHMWPE) and is shaped like a dome to the surface of the talus part and matched to the interior concavity of the stem base.

As further illustrated in FIG. 6B, there is indicated an oblique resection R at the top end of the anterior cortex window, which will be discussed in a procedure related to implantation hereinafter.

As noted, FIGS. 7A through 7Q illustrate one possible example embodiment of an implantation procedure for the TAR 100 depicted in FIGS. 6A-6C.

FIG. 7A shows an anterior view of the native configuration of the tibia 10 and fibula 12, including the relative locations of the inner cortex 18 and outer cortex 16.

In a first step, illustrated in FIGS. 7B and 7C, two drill holes are made in the distal end of the tibia 10. FIG. 7B shows an anterior view of the tibia 10 with two drill holes 108A and 108B that have been made from the front to the back to create an anterior window for eventual tibial component placement. The drill hole 108A is a proximal large drill hole, whereas the drill hole 108B is a distal larger drill hole. Alternatively, this same bone resection can be completed with multiple perforations from a drill bit, burr, saw or other tool. FIG. 7C shows a lateral view of the drill holes 108A and 108B seen in FIG. 7B. The path of the distal larger drill hole 108B is parallel to the end of the tibia 10. The path of the smaller more proximal drill hole 108A is aimed inferiorly and posteriorly. The top surface 106 of the talus 14 is also resected. The lateral aspect of the talus 14 after a flat-cut is also depicted in FIG. 7C.

Next, FIG. 7D shows a curved awl 110 used to penetrate the tibial canal 11 to create a space for subsequent broaching. FIG. 7E shows deeper penetration of the awl 110 into the tibial canal 11. The awl 110 is curved and designed to avoid penetration of the cortex and only stay within the softer tibial canal 11.

After creating enough space with the awl 110, FIG. 7F shows a small entry broach 112 being placed into the tibial canal 11 to form a receiving cavity for the stem part 42 of the stem part 40. The broach 112 is shaped similar to the stem part 42 of the stem part 40 but is smaller than the definitive size. Optionally, one or more broaches 112 of increasing size may be used sequentially to shape and enlarge the receiving cavity into the tibial canal 11. FIG. 7G shows the final broach 112 placement after sequentially larger broaches have been used to enlarge the receiving cavity. This final broach 112 has sufficient press-fit in the tibial canal 11. The size of the final broach 112 is then used to determine the appropriate size of the tibial implant 40.

Next, excess bone can be removed from the tibia 10 and the fibula 12 if needed. FIG. 7H shows a targeting jig 114 attached to the final broach 112. This targeting jig 114 can be used to guide a burr, saw or other bone machining tool to remove excess bone from the tibia 10 and fibula 12. FIG. 7I shows proposed cuts 116a, 116b, and 116c made off the fibula and tibia to complete the bone resection needed to allow for implant placement. FIG. 7J shows an anterior view of the resected tibia 10 and fibula 12 bones after the cuts 116a-c have been made with the final broach 112 (and the targeting jig 114) removed from the tibia 10. Preferably, the edges of these resections are smoothed for optimal low stresses. This is in contradistinction to standard conventional TAR installation procedures which form sharp corners C as shown in FIG. 5A. FIG. 7K shows a lateral view of the tibia 10 and talus 14 after bone resection. The talus is in the “neutral” position at approximately 90 degrees to the axis of the tibia 10 in this view.

The talus 14 is then flexed in the plantar direction to provide sufficient clearance between the tibia 10 and the talus 14 for insertion of the stem part 40. FIG. 7L shows a lateral view similar to FIG. 7K but with the talus plantarflexed (downward) to create space for tibial component placement.

Next, the stem part 40 is inserted into the receiving cavity within the tibial canal 11. FIG. 7M shows a lateral view of penetrance of the stem part 40 in the tibial canal 11 entering anteriorly and inferiorly. The end 44 is inserted into the tibial canal 11 such that the base 70 will extend from the distal end of the resected tibia 10. FIG. 7N shows a similar view to FIG. 7M as the stem part 40 is placed (partially inserted) further into the tibial canal 11 and rotates distally to better approximate the axis of the tibia 10.

FIG. 7O shows a similar view to FIG. 7N with the stem part 40 definitively seated in the tibia 10 and press-fit into the tibial canal 11 in its final intended implant position.

Typically, although not necessarily thereafter, the talus part 50 is then attached to the flattened top surface of the talus 14, and the hemispherical top surface of the talus part 50 is seated within the concave (lower) surface of the liner 52, and the convex (upper) surface of the liner 52 is seating within the concavity 48 in the lower surface of the base 70 of the stem part 40. FIG. 7P shows a lateral view of the ankle with definitive placement of all components of the prosthetic ankle replacement (TAR) 100 similar to FIG. 6B. FIG. 7Q shows an anterior view of the ankle with definitive placement of all components of the TAR 100, along with a dotted line representing the bone resections made for implantation. This represents superimposing of FIGS. 6A and 7K.

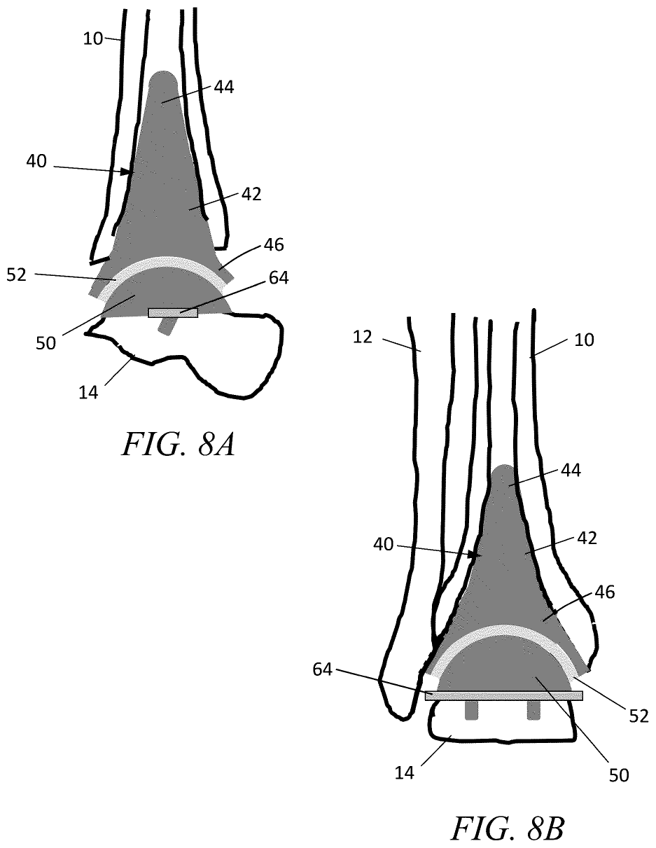

FIGS. 8A and 8B illustrate one or more projections, or extensions, may also be provided on the talus component itself. Such projections 64 extend generally radially from the circumference of the talus component, most preferably from the lower circumference. The projections perform the function of stops in essence, to prevent the tibial component from sliding in undesirable inversion or eversion.

Optionally, the stem part 40 and the talus part 50 may be advantageously made with a sintering process and/or with an additive manufacturing 3D printing, to have connected porosity within one or both of these parts 40/50. This may allow for the formation of channels 72 therein (shown schematically in FIG. 7P), which allow diffusion of fluid within the part 40/50, and therefore to more function like a bone under transient dynamic loading conditions. At least some of the channels 72 extend from an exterior of the respective stem part 40 and/or talus part 50 into an interior thereof. These channels 72 allow diffusion of fluid such that the part 40/50 will function like a bone under transient dynamic loading conditions. This will result in distribution of load over the tibial and talus parts 40 and 50 minimizing the dynamic stresses, and hence life.

The TAR 100 and related implementation methods of the present invention are believed to overcome existing deficiencies in conventional TARs by creating robust contact between a tibia stem and the inside surface of the tibia cortical bone. This allows improved axial load transfer with lower stress levels imposed on the tibia 10 and talus 14.

Assembly tools and procedures are provided to improve alignment and preload accuracy on the stem to distribute the load over a large area, as depicted variously in FIGS. 7A-7Q. The technique of osteotomy, canal entry, and sequential broaching mimics the standard technique for hip replacement femoral stem placement, but it is considered to be new and unconventional as provided to ankle replacement. In order to place the tibia part 40, a cavity is gradually created through controlled broaching to accept the top surface of the stem. Broaching and tibia device placement may be done manually or using a computer-controlled system with load sensors for another technique.

It is expected that keeping stresses below the endurance limit will result in enhanced life of the TAR 100 relative to conventional ankle replacements prostheses. Improved alignment also helps with longevity. Nanomaterials may be utilized in the parts design to improve boundary lubrication. Microscopic passages (e.g., channels 72) may be provided through the upper stem and the top side to allow fluid migration to lubricate the sliding surfaces (see, e.g., FIG. 6C).

In a nonlimiting example, the design process for a tibial stem (tibial part) 40 involves obtaining the geometry of the tibia. From the geometry of the tibia bone's external surface an estimate of the internal cortical part of the bone could be obtained. From the inside geometry of the tibia, a tentative shape of the stem part 40 (stem) may be generated. This shape can be analyzed for its loadbearing capacity. If found to be sufficiently strong, a graphical means (or computerized equivalent means) could be used to determine the ability to assemble the stem into the tibia. During assembly, known processes could be incorporated to measure the applied axial force to calculate the measured radial interference. From the bone properties, the adequacy of the interference may be evaluated.

The components of the TAR 100 may be manufactured by any effective means. While not exclusive, it is considered most preferred that all metallic components of the inventive TAR (e.g., the stem part 40 and talus part 50) are made by a sintering method wherein powdered material (of graded particle size distribution) is used to make a part in ‘green state’ by compressing the powder in a mold. The green part is then heated in a furnace to the sintering temperature and held for a required length of time. The sintered metal and plastic materials have lower mechanical strength but are stronger than the bones. They also have a lower Young's modulus (compared to the parent material) which tends to make their compressive behavior similar to that of bones for a given size. This helps distribute load more uniformly over the contact area between the tibia and talus. Manufacturing may also be done by 3D printing (additive manufacturing) to achieve similar characteristics. Standard coating and porous surfaces may also be created to allow for bone ingrowth or cement fixation.

The spherical joint between the liner 52 and metallic talus part 50 may be made with projections 62 at the periphery of the plastic component to slow escape of synovial fluid during axial shock loading. Based on the amount of fluid between the components, the projections 62 may or may not be necessary.

There are numerous advantages in the new prosthetic bone joint replacement implant (e.g., TAR 100) relative to conventional joint replacement prostheses. For example, the large articulation contact area of the new prosthetic bone joint replacement implant (e.g., TAR 100) will increase the usable life span of the replacement joint due to larger contact area for load transfer from tibia to talus through the spherical joint. The fluid pressure (or equivalently the mechanical stress) in the bearing area is reduced due to the larger contact area. The lower stress level should increase the boundary layer thickness which should reduce the wear rate in the sliding parts. In some arrangements, mechanical subsidence of the tibia metal component may be reduced due to the larger contact area from the press-fit tibia stem component as compared to the traditional end-loading tibia components. In some arrangements, the TAR may have improved performance in that it should allow a patient to perform higher impact activities as compared to traditional TARs. In some arrangements, reduced friction and increased mass in the TAR joint will lead to lower transient temperatures (in the sliding joint) keeping it below 42° C., and thus reduce the degradation of synovial fluid and prevent solids precipitation from the synovial fluid at high transient temperatures (e.g., T>42° C.) in the sliding joint. In some arrangements, the ball-and-socket joint design of the new TAR 100 is based on a spherical (ball and socket) joint allowing angular displacements of the required magnitudes about two orthogonal axes. This makes the design more tolerant to angular misalignment of tibia to talus, preventing wear due to edge loading. It also provides protection against shock loads. Additionally, the spherical design allows for inversion and eversion movement to further offload adjacent joints. In some arrangements, the length and diameter of the stem will be designed to result in a press-fit with the tibial canal, which will ensure improved load transfer capability through the cortical section of healthy proximal tibia. The tibia component will not rely on arthritic distal tibial bone for support. In some arrangements, a cushioning effect may be achieved, for example, because articulation will be designed to maintain a sufficient boundary layer for cushioning between the plastic and metal talus components during axial shock loading. A plastic component may advantageously include peripheral projections to retain sufficient synovial fluid between components.

According to some nonlimiting aspects of the invention, stem geometry of a tibial element implant has been optimized to facilitate implant installation while providing improved means to transfer load over a larger surface area through a “press-fit,” “scratch-fit” or “cortical fit,” thus lowering the contact stresses at the tibia-metal implant interface. The design and its implantation procedure also address minimization of misalignment.

As previously noted above, though the foregoing detailed description describes certain aspects of one or more particular embodiments of the invention, alternatives could be adopted by one skilled in the art. For example, the joint replacement prosthesis, such as the TAR 100, and its components could differ in appearance and construction from the embodiments described herein and shown in the drawings, functions of certain components of the joint replacement prosthesis could be performed by components of different construction but capable of a similar (though not necessarily equivalent) function, and various materials could be used in the fabrication of the joint replacement prosthesis and/or its components. As such, and again as was previously noted, it should be understood that the invention is not necessarily limited to any particular embodiment described herein or illustrated in the drawings.

This methodology as well as the prosthesis itself, as set forth for ankle replacement could be used in other applications such as hip, knee or shoulder replacements. Also, increasing the size of the elements may further adapt the prosthesis to be used for other applications such as when a patient has lost part of their bone in an accident or the talus bone has lost its blood supply.

Although the devices, components, materials and methods have been described and illustrated in connection with certain embodiments, many variations and modifications will be evident to those skilled in the art and may be made without departing from the spirit and scope of the disclosure. For instance, while the invention has found particular form for ankle replacement, aspects may nevertheless be applicable to other joints in the body. The disclosure is thus not to be limited to the precise details of methodology or construction set forth above as such variations and modification are intended to be included within the scope of the disclosure, and the Claims hereafter.

What is claimed is:

1. A prosthetic ankle replacement comprising:
a talus element which is to be affixed to the surface of the talus, the talus element presenting a partial-hemispherical surface;
a tibial element which is to be affixed within the tibia, the tibial element having an elongated stem part which extends into and within a canal of the tibial inner cortex, an intermediate body part which smoothly widens in radius toward an end which is a base to the tibial element, the base having a concavity formed therein shaped to match the partial-hemispherical surface of the talus element;
the tibial element being adapted to conform to a shape formed in the tibia so as to be press fit into the tibial canal with a designed axial load.

2. The prosthetic ankle replacement of claim 1, further including a hard plastic cap component which provides an interface between the talus element partial-hemispherical surface and the tibial base concavity.

3. The prosthetic ankle replacement of claim 2, wherein the hard plastic cap is formed as a hemispherical dome and further including a projection at a periphery to the cap to limit the outward flow of synovial fluid exiting a space between the plastic cap and metal talus component in order to maintain a cushioning boundary layer under dynamic impact loads.

4. A method for implantation of a prosthetic ankle replacement, comprising:
providing a prosthesis of a talus element which is to be affixed to the prepared surface of the talus, the talus element presenting a partial-hemispherical surface; a tibial element which is to be affixed within the tibia, the tibial element having an elongated stem part which extends into and within a canal of the tibial inner cortex, an intermediate body part which smoothly widens in radius toward an end which is a base to the tibial element, the base having a concavity formed therein shaped to match the convex surface of the hemispherical hard plastic cap while the partial-hemispherical surface of the talus element matches the concave surface of the hemispherical hard plastic cap;
cutting an end of the tibia to expose the inner tibial canal, and successively widening the canal through removal of intramedullary bone therein so as to match the external shape of the tibial element;
affixing the talus element to the prepared surface of the talus so as to present the partial-hemispherical surface toward the concavity of the tibial element;
press fitting the tibial element into the tibial canal with a designed axial load; and
seating the talus element within the concavity of the hard plastic cap while seating the hard plastic cap within the concavity of the tibial element.

5. The prosthetic ankle replacement of claim 1, wherein at least one of the tibial and talus elements are made using a sintering process or an additive manufacturing layer wise build process to have connected porosity from channels extending from an exterior of the element and into the element to allow diffusion of fluid to function like a bone under transient dynamic loading conditions.

6. A prosthetic for implantation in a joint of a human or animal comprising:
a first element which is to be affixed to the surface of a first end of a joint, the first element presenting a partial-hemispherical surface; and
a second element which is to be affixed within an second end of the joint opposed to the first end, the second element having an elongated stem part which extends into and within a canal of the second end of the joint, an intermediate body part which widens in radius toward an end which is a base to the second element, the base having a concavity formed therein shaped to match the partial-hemispherical surface of the first element;
the second element being adapted to conform to a shape formed in the canal so as to be press fit into the canal.

7. The prosthetic of claim 6, further including a hard plastic cap component which provides an interface between the first element partial-hemispherical surface and the second element base concavity.

8. The prosthetic of claim 6 wherein the first element is adapted for a talus element, and the second element is adapted for a tibial element which is received within a canal of a tibial inner cortex.

9. The prosthetic of claim 8, further including a hard plastic cap component which provides an interface between a talus element partial-hemispherical surface and a tibial base concavity.

10. A prosthetic for implantation in a joint of a human or animal, the joint including a bone having a cortical canal, comprising:
a stem part which has a long axis and a radial aspect, the stem part having a distal end which extends into and within the canal of the canal in use, an intermediate body part which widens in radius toward a proximal end which is a base to the stem part,
the stem part being adapted to present a stem exterior that conforms to a shape formed in the canal yielding an open canal interior sidewall which is matched to the radius of the stem, so as to be press fit into the canal and held in place through contact between the canal interior sidewall and the stem exterior.

11. The prosthetic of claim 10, wherein the stem part is affixed without any use of cement or other mechanical fixation.

12. The prosthetic of claim 10, further including a first element which is to be affixed to the surface of a first end of a joint, the first element presenting a partial-hemispherical surface; the intermediate body part which widens in radius toward an end which is the base wherein the base has a concavity formed therein shaped to match the partial-hemispherical surface of the first element.

13. The prosthetic of claim 10, further including a hard plastic cap component which provides an interface between the first element partial-hemispherical surface and the base concavity.

14. The prosthetic of claim 13, wherein the first element is adapted for a talus element, and the stem part is adapted for a tibial element which is received within a canal of a tibial inner cortex.

15. The prosthetic of claim 14, further including a hard plastic cap component which provides an interface between a talus element partial-hemispherical surface and a tibial base concavity.

16. A prosthetic ankle replacement comprising:
a talus element which is to be affixed to the surface of the talus, the talus element presenting a partial-hemispherical surface; and
a tibial element which is to be affixed within the tibia, the tibial element having an elongated stem part which extends into and within a canal of the tibial inner cortex, an intermediate body part which widens in radius toward an end which is a base to the tibial element, the base having a concavity formed therein shaped to match the partial-hemispherical surface of the talus element;
the tibial element being adapted to conform to a shape formed within the tibia presenting tibial cortex canal sidewalls so as to be press fit into the tibial cortex canal and affixed in place without further means of fixation.

17. The prosthetic ankle replacement of claim 16, further including a hard plastic cap component which provides an interface between the talus element partial-hemispherical surface and the tibial base concavity.

18. A method for implantation of a prosthetic bone joint replacement, comprising:
providing a prosthesis of a first element which is to be affixed to the surface of an end of a first bone of the joint, the first element presenting a partial-hemispherical surface; a second element which is to be affixed within a cortical canal of a second bone of the joint, the second element having an elongated stem part which extends into and within a canal of the cortical canal, an intermediate body part which widens in radius toward an end which is a base to the second element, the base having a concavity formed therein shaped to match the partial-hemispherical surface of the first element;
cutting an end of the second bone to expose the inner canal, and successively widening the canal through removal of intramedullary bone therein so as to match the external shape of the second element;
affixing the first element to the surface of the first bone so as to present the partial-hemispherical surface toward the concavity of the second element;
press fitting the second element into the canal into tight engagement with a sidewall forming the interior of the canal; and
seating the first element within the concavity of the second element.

19. The method of claim 18, wherein the second element is affixed without any use of cement or other mechanical fixation.

20. The method of claim 19, further including providing a hard plastic cap component which provides an interface between the first element partial-hemispherical surface and the base concavity.

21. The method of claim 20, wherein the first element is adapted for a talus element, and the stem part is adapted for a tibial element which is received within a canal of a tibial inner cortex.

22. The method of claim 21, further including a hard plastic cap component which provides an interface between a talus element partial-hemispherical surface and a tibial base concavity.

23. The prosthetic ankle replacement of claim 16, wherein the tibial element is made as a single piece.

24. The prosthetic of claim 10, wherein the stem part made as a single piece.

25. The prosthetic of claim 6, wherein the second element is made as a single piece.

26. The prosthetic ankle replacement of claim 1, wherein the tibial element is made as a single piece.

27. The method of claim 4, wherein the tibial element is made as a single piece.

28. The method of claim 18, wherein the second element is made as a single piece.

29. The prosthetic ankle replacement of claim 16, wherein the talus element further includes projections extending outwardly from the talus element, the projections adapted to limit inversion and eversion motion of the prosthesis.

30. The prosthetic of claim 12, wherein the first element further includes projections extending outwardly from the talus element.

31. A prosthetic ankle replacement comprising:
a talus element which is to be affixed to the surface of the talus, the talus element presenting an articulation upper interface surface; and
a tibial element which is to be affixed within the tibia, the tibial element having an elongated stem part which extends into and within a canal of the tibial inner cortex, an intermediate body part which widens in radius toward an end which is a base to the tibial element, the base having a shape formed thereon shaped to interface with the articulation upper surface of the talus element;
the tibial element being adapted to conform to a shape formed within the tibia presenting tibial cortex canal sidewalls so as to be press fit into the tibial cortex canal and affixed in place through such press fit.

32. The prosthetic ankle replacement of claim 31, wherein the tibial element is affixed without further means of fixation.

33. The prosthetic ankle replacement of claim 31, wherein the tibial element base has a concavity formed therein shaped to match a partial-hemispherical surface of the talus element.

34. The prosthetic ankle replacement of claim 31, wherein the articulation upper interface surface is a hard plastic component provided on the talus element upon which the tibial element is seated.

35. The prosthetic ankle replacement of claim 31, wherein the talus element further includes projections extending outwardly from the talus element, the projections adapted to limit inversion and eversion motion of the prosthesis.

36. A prosthetic ankle replacement comprising:
a talus element configured to be affixed to the surface of the talus, the talus element presenting a partial-hemispherical surface; and
a tibial element configured to be affixed within the tibia, wherein the tibial element comprises an elongated stem part sized and shaped to extend into and within a canal of a tibial inner cortex, an intermediate body part which smoothly widens in radius toward an end which is a base to the tibial element, the base having a concavity formed therein shaped to match the partial-hemispherical surface of the talus element;
wherein the tibial element is adapted to conform to a shape formed in the tibia so as to be press fit into a tibial canal of the tibia with a designed axial load.

37. The prosthetic ankle replacement of claim 36, further comprising:
a hard plastic cap component which provides an interface between the partial-hemispherical surface of the talus element and the concavity formed in the base of the tibial element.

38. The prosthetic ankle replacement of claim 37, wherein the hard plastic cap component is formed as a hemispherical dome and further comprises a projection at a periphery of the hard plastic cap component to limit the outward flow of synovial fluid exiting a space between the hard plastic cap component and the talus element in order to maintain a cushioning boundary layer under dynamic impact loads, and wherein the talus is metal.

39. The prosthetic ankle replacement of claim 36, wherein the tibial element is made as a single piece.

40. A method for implanting a prosthetic ankle replacement, the method comprising:
providing prosthetic ankle replacement including a talus element, a hemispherical hard plastic cap, and a tibial element, wherein the talus element is configured to be affixed to a prepared surface of the talus, the talus element presenting a partial-hemispherical surface that forms a convex surface, and wherein the tibial element is configured to be affixed within the tibia, the tibial element having an elongated stem part which is configured to extend into and within a tibial canal of the tibial inner cortex, an intermediate body part which smoothly widens in radius toward an end which is a base to the tibial element, the base having a concavity formed therein shaped to match a convex surface of the hemispherical hard plastic cap while the partial-hemispherical surface of the talus element matches a concave surface of the hemispherical hard plastic cap;
cutting an end of the tibia to expose the tibial canal, and successively widening the tibial canal through removal of intramedullary bone therein so as to match an external shape of the tibial element;
affixing the talus element to the prepared surface of the talus so as to present the partial-hemispherical surface toward the concavity of the tibial element;
press fitting the tibial element into the tibial canal with a designed axial load; and
seating the talus element within the concave surface of the hard plastic cap while seating the hard plastic cap within the concavity of the tibial element.

41. The method of claim 40, wherein the tibial element is made as a single piece.

42. A prosthetic ankle replacement comprising:
a talus element to be affixed to a surface of a talus, the talus element presenting a partial-hemispherical surface; and
a tibial element to be affixed within a tibia, the tibial element having an elongated stem part configured to extend into and within a canal of an inner cortex of the tibia, an intermediate body part that widens in radius toward an end which forms a base of the tibial element, the base having a concavity formed therein shaped to match the partial-hemispherical surface of the talus element;
the tibial element being adapted to conform to a shape formed within the tibia presenting canal sidewalls of the canal of the inner cortex of the tibia so as to be press fit into the canal of the inner cortex and affixed in place without further means of fixation.

43. The prosthetic ankle replacement of claim 42, further including a hard plastic cap component which provides an interface between the partial-hemispherical surface of the talus element and the concavity of the base of the tibial element.

44. The prosthetic ankle replacement of claim 42, wherein the tibial element is made as a single piece.

45. A method for implantation of a prosthetic bone joint replacement, the method comprising:
providing a prosthesis comprising: a first element which is to be affixed to the surface of an end of a first bone of the joint, the first element presenting a partial-hemispherical surface; and a second element which is to be affixed within a cortical canal of a second bone of the joint, the second element having an elongated stem part which extends into and within the cortical canal, an intermediate body part which widens in radius toward an end which forms a base to the second element, the base having a concavity formed therein shaped to match the partial-hemispherical surface of the first element;
cutting an end of the second bone to expose the cortical canal;
successively widening the cortical canal through removal of intramedullary bone therein so as to match an external shape of the second element;
press fitting the second element into the cortical canal into tight engagement with a sidewall forming an interior of the cortical canal;
affixing the first element to the surface of the first bone so as to present the partial-hemispherical surface toward the concavity of the second element; and
seating the first element within the concavity of the second element.

46. The method of claim 45, wherein the second element is affixed without any use of cement or other mechanical fixation.

47. The method of claim 46, further including providing a hard plastic cap component which provides an interface between the partial-hemispherical surface of the first element and the concavity of the base.

48. The method of claim 47, wherein the first element forms a talus element, and the stem part forms a tibial element which is received within the cortical canal of a tibial inner cortex.

49. The method of claim 48, wherein the hard plastic cap component provides an interface between the partial-hemispherical surface of the talus element and the concavity of the base.

50. The method of claim 49, wherein the second element is made as a single piece.
